(19) United States
(12) Patent Application Publication
(10) Pub. No.: US 2023/0197229 A1
(43) Pub. Date: Jun. 22, 2023

(54) Compounding application ordering system

(71) Applicant: Sameh Mohamed, Calgary (CA)

(72) Inventor: Sameh Mohamed, Calgary (CA)

(21) Appl. No.: 17/553,826

(22) Filed: Dec. 17, 2021

(51) Int. Cl. G16H 20/10; G16H 40/67; G16H 70/40

(52) U.S. Cl. CPC G16H 20/10; G16H 40/67; G16H 70/40

(57) ABSTRACT

This disclosure relates generally to a system and method for the optimized management of pharmacy workflow, specifically, workflow associated with fulfilment of compounding prescription orders. A computer-controlled system coordinates and controls workflow to receive, sequence, process and track prescriptions for fulfilment in a most efficient path thereby minimizing errors and providing prompt output. The software is a price estimator and an ordering system for compounded medications. When a retail pharmacy receives a prescription for a medication that needs to be compounded, they can use the software to enter ingredients and other components to get a price quote and consequently order the compound from a specialized compounding pharmacy. A smartphone, a laptop computer, a portable communicating device, a cloud computing device, or a data storage device participate in the process. The proposed method reduces errors and provides quick, clear, and efficient communication and feedback/follow up features. In order words, it makes a compounding medicines assessment, order placement, fulfilment and status tracking a breeze.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever

### BACKGROUND

### Field of the Invention

Embodiments relate to an pharmacy data processing systems and more particularly to data processing systems used to manage price estimation and ordering system of compound medicines. The present invention further relates to methods and an apparatus for developing, analyzing, calculating, supporting and advising pharmacy-related decisions.

### Description of the Related Art

Out of approximately 56,000 U.S. pharmacies, about 7,500 specialize in pharmaceutical compounding. As such, these pharmacists spend a great deal of time preparing special medications and treatments for patients. Due to the nature of compounding, pharmacists who specialize in this practice are closely regulated by federal and state authorities. Drug compounding is a process by which a pharmacist or doctor combines, mixes, or alters ingredients to create a medication tailored to the needs of an individual patient. Compounding is typically used to prepare medications that are not commercially available. Compounders may swap out an ingredient that a patient may be allergic to or may change the dosage form into a more suitable form (e.g., taking a large tablet and making the drug product into a powder than can be swallowed easier).

Before the prevalence of mass-produced drugs, this was a very common practice, but now it has largely been relegated to special cases where a mass-produced version of a drug is either unavailable or unsuitable for a patient. Provided that each compounded medication is uniquely prepared for an individual patient pursuant to a physician's order, compounding generally falls outside the jurisdiction of the FDA and instead within the regulation of the practice of Pharmacy under relevant state law. This line is not always clear, but there is strong general support for maintaining the flexibility needed to personalize medication at the pharmacy level and likewise not unduly bias physicians towards prescribing mass-produced products or dosage levels. But if the “compounding” is not for just one patient/customer but prepared in anticipation of multiple similar prescriptions, it may cross the line into “manufacturing”—and thus subject to such regulations.

Most compounding is either manual or partially-automated with the help of certain tools or equipment. So-called Automated Compounding Devices (ACDs), having full automation, exist at this time only for parenteral/intravenous (I.V.) medications, which are prepared as comparatively high-volume liquid solutions administered from bags (typically in a hospital).

There are multiple inventions that have been proposed in prior art regarding pharmacy compounding. However, the utility of these systems has not been seen in advance form and for specialized needs. Furthermore, with the advancement in market, more advance methods are needed to facilitate the compounding process.

Currently most existing and traditional pharmaceutical method provide manual process of compounding where the patient brings the prescription to pharmacy which mixes the required dosage of multiple medicines to produce one medicine. However, this method is manual and requires more attention and is prone to mistakes. Furthermore, in the context of price calculations accurate and instant pricing is also a time-consuming process. Sometimes, it results in a output which is not cost-effective, clinically-correct or safe.

The current invention proposes a system where a new approach, utilizing the best of breed in existing technologies, to revolutionize the digital arena, implementing the bridge that fills the gap between pharmacy, hospitals and patients is implemented.

The proposed platform seeks to significantly simplify the whole process and efficiently manages the circumstances through its swift ability to digitalize the whole process. The invention relates to the development of an automated compounding system that addresses the needs and desires of both healthcare providers (e.g., clinicians, pharmacists and health professionals) and patients. The system encompasses computer communications network-based systems, software, various input and output stations in order to provide desired output. The system also aims to presents multiple additional features which can provide advance level of ease in drug compounding calculations, errors elimination, instant price quotation, ability to receive, track and delivery insurance of final order.

As per applicants' knowledge, none of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is therefore the purpose of the invention to alleviate at least to some extent one or more of the aforementioned problems of the prior art and/or to provide the relevant public with a suitable alternative thereto having relative advantages.

The primary object of the invention is related to the provision of an automated drugs compounding system that addresses the needs and desires of both healthcare providers and patients. The system encompasses computer communications network-based systems, software, various input and output modules for provision of an advance level of drug compounding application.

It is also the objective of the invention to provide a simplified process of automated drugs compounding system including an input and output module. The input module linked with back-end pharmacy database is adapted to store the pharmacy order received. The pharmacy database is adapted to retrieve the required context data from the record in the information database, and provide the output in form of automatic calculation of inventory, price quotation and required dosage of medicine.

It is moreover the objective of the invention to provide a system which providing a method of eliminating the most of the errors that occur during a manual order placement process.

It is also the objective of the invention to provide a system where provision of an instant price quotation is also provided. Furthermore, options of courier services and other indicators can also be selected.

It is moreover the objective of the invention to provide a platform, where order can be received by phone, by image received at the portal and can be input by application interface, processed and tracked through the application.

It is further the objective of the invention to provide a dual and reliable system which is easy to install and is easily compatible for all types of smart devices and provides secure electronic transmission.

It is moreover the objective of the invention to provide a system which is inline with latest technologies and utilizes the latest health care information system including but not limited to real time environment, a highly compatible and artificial intelligence-based system is introduced.

It is further the objective of the invention to provide an interface which is easy to use, and where new users are provided with tutorials and necessary guidance to use the system.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner. The current invention in its preferred embodiment discloses a new approach, toward pharmacy compounding system.

The current invention as per its preferred embodiments provides a system which has a main focus on approaches relevant with pharmacy decisions, where assessment and timing is a complex process, particularly when viewed in the context of the fact that the process is very sensitive and little error can result in bigger problems. The current invention as per its preferred embodiments discloses a quick, accurate, retrievable and convenient approach to provide the best outcome of care and maximum safety of the patient. This scenario becomes excessively intricate when personnel, resources are scarce and time pressure is ever mounting. Under those conditions, there is need to develop a system which allows for seamless, efficient, and accurate information management which is readily retrievable and is amenable to intervention, modification and reassessment up and down the hierarchical of providers. Recent advances in the communication industry, particularly immense computing power of handheld devices have offered an ample playing field to fabricate systems for specific and varied purposes. The current invention takes into account all those aspects and addresses a critical need for desired and essential collaboration in pharmaceutical care.

In embodiments, methods and systems for automatically receiving, maintaining and tracking the compounding order in accordance with various embodiments of the present invention may be provided. In embodiments, methods and systems may include an automatic data collection facility which refers to a server-based system compromising of a data receiving module configured to receive data from the patients.

The pharmacy compounding system features, which are further elaborated and defined for reference here. In this embodiment, the application consists of multiple screens. Screens are labelled by the indication or by dosage form (Ex Topical Pain Killers, Capsules etc.). The Client's (mostly retail pharmacy) profile could be as one of the main screens or on a side menu.

The first screen as per its preferred embodiments is dedicated for Topical Pain Killers. It consists of 11 components. 10 components for data entry and one field for price viewing. The 10 components include active Ingredients which is a scroll wheel where all the active ingredients are listed like Gabapentin, Diclofenac. The client can choose multiple ingredients to be incorporated in the same compound. Price for each ingredient is set in the background.

A Scroll wheel as per its further embodiments is where all the bases are listed. The client can choose the base that the ingredients will be incorporated into. Price for each base is set in the background. Furthermore, a scroll wheel with a various quantity range. The client can choose the total quantity of the compounded medication to be ordered. Quantity tab could be eliminated if ordering 100 gms which is the most common.

A scroll wheel with Packaging is also provided. The price of every type of packaging is set in the background. Also, a scroll wheel with delivery to various destinations. Price per destination is set in the background. A calculate price button to calculate the price of the compound according to all the prices set in the background as per the above inputs.

The price field is also provided for the client to view and confirm the price of the compound before ordering. A picture module to take picture of the prescription is also provided. Sometimes the prescription is for a complex compound or sometimes there's ambiguity with the compound instructions. The retail pharmacy personnel can take a picture of the compound and consult the compounding pharmacist to ensure a proper and safe compound is made.

A send button to send the compound order or a picture of it to the compounding pharmacy in timely manner. Also, a note field to send any additional information from the retail pharmacy to the compounding pharmacy is also provided. The phone number to call the compounding pharmacy in case if a consultation is needed is also provided.

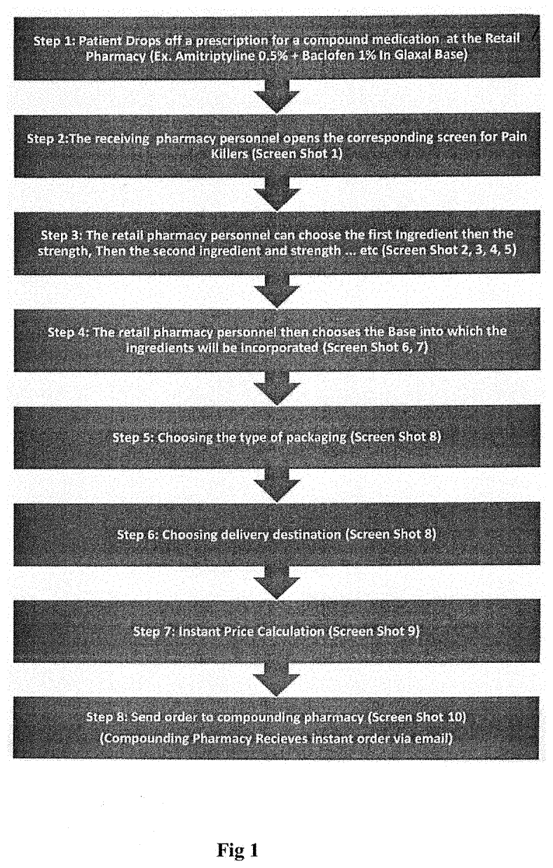

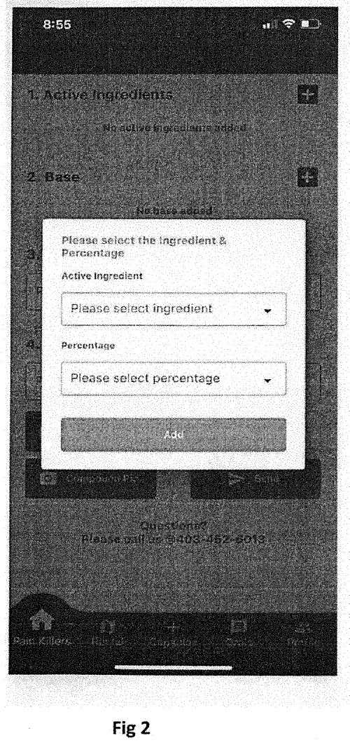

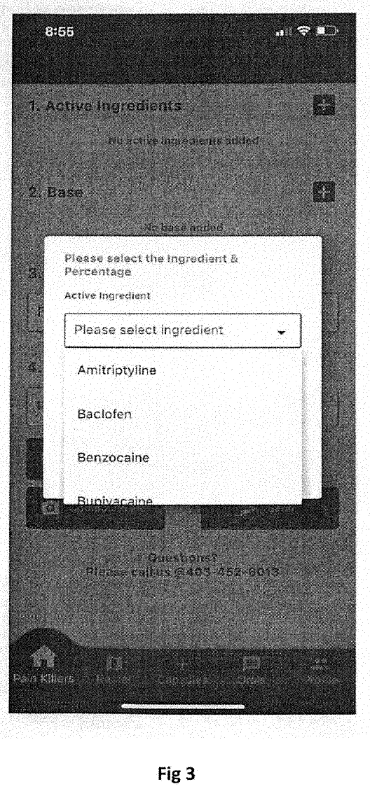

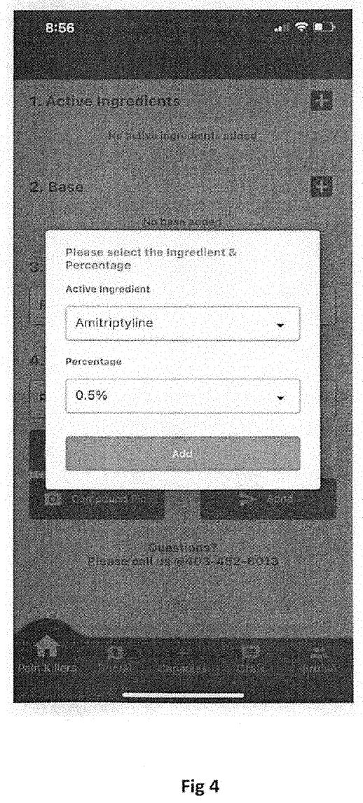

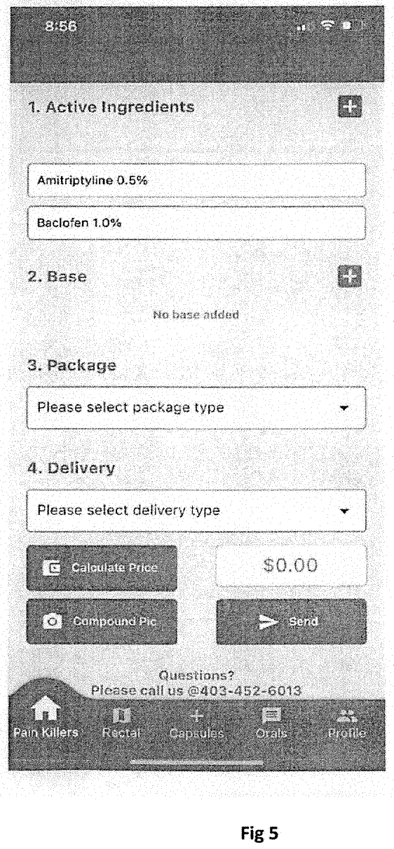

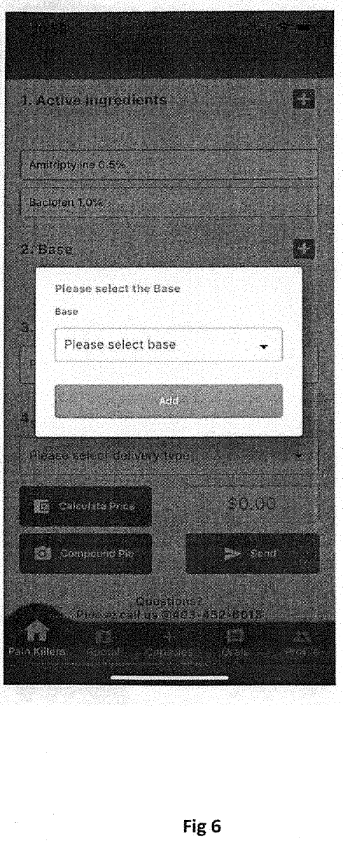

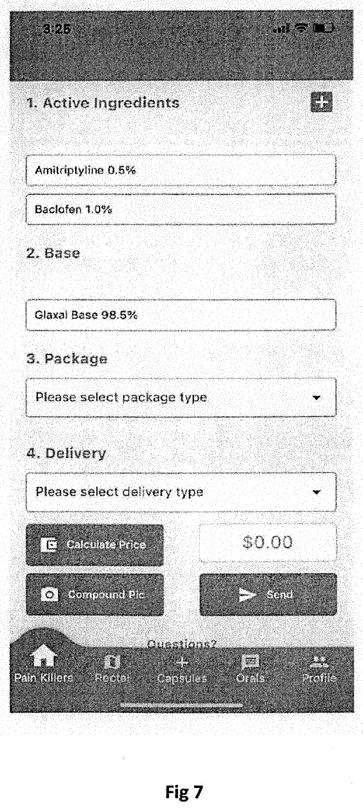

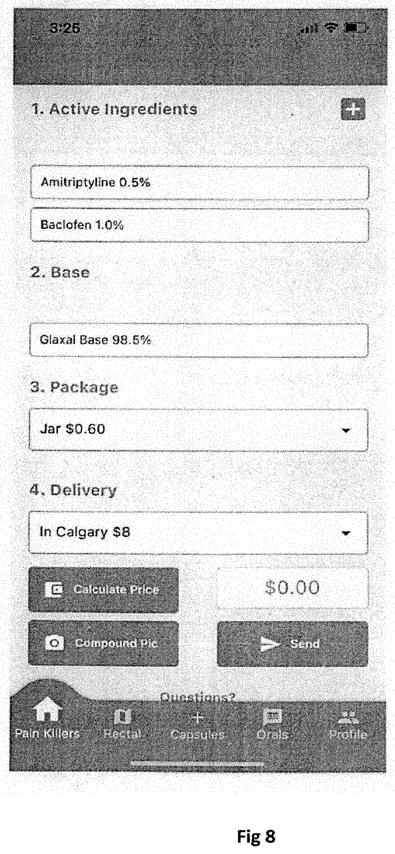

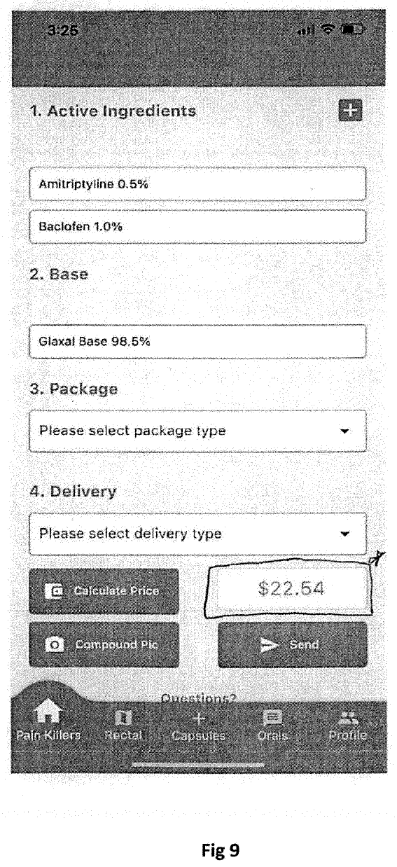

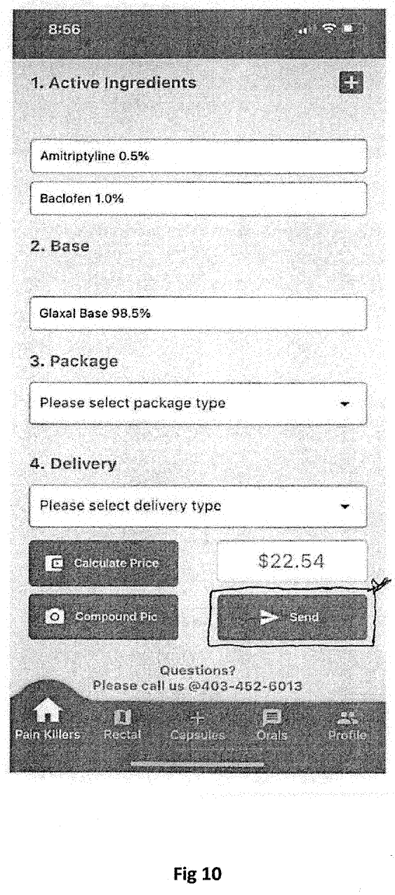

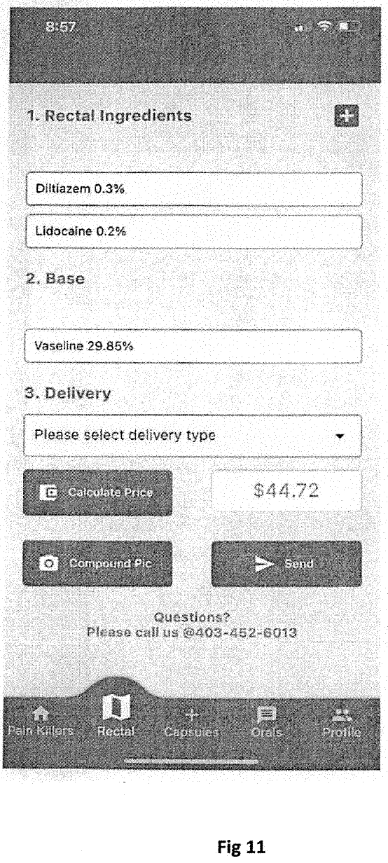

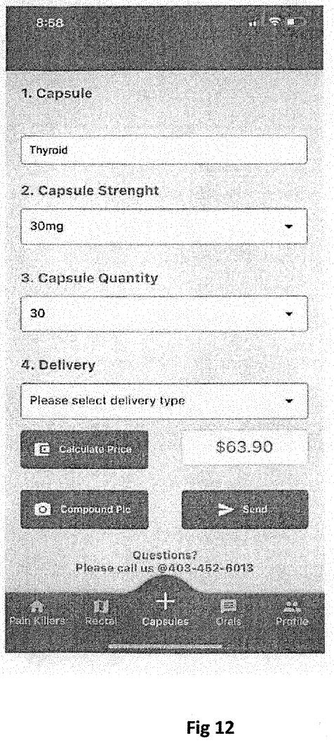

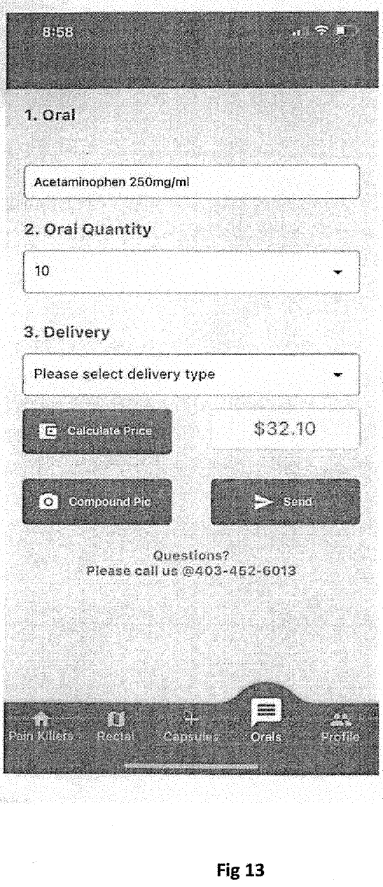

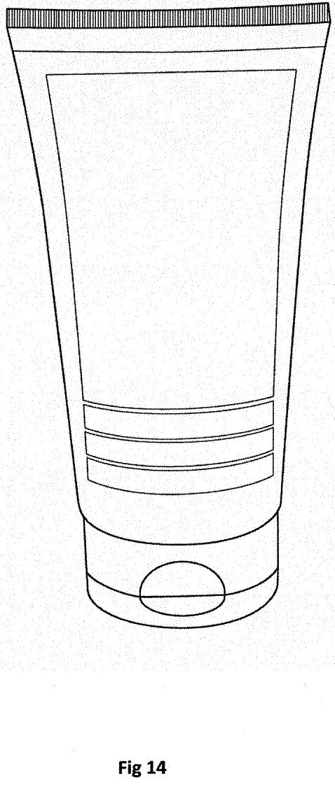

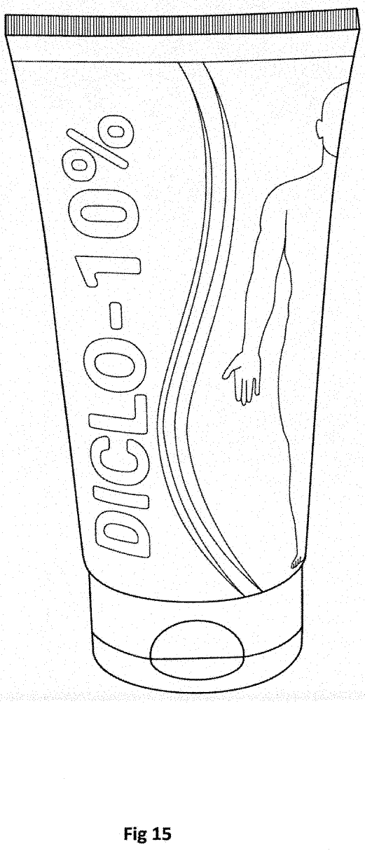

By looking at provided images multiple modules of application can be seen and understood. FIG. 1 shows the process flow as per preferred embodiments. FIG. 2-13 are showing multiple views and configurations provided where as other screens could be added according to market needs (Ex. Hormones, Dermatological compounds, Sterile compounds etc). FIGS. 14 & 15 are showing the final output.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1: A pharmaceutical drug compounding system to create, process transmit, track, a pharmaceutical care order, the system comprising: a plurality of service application instances, devices under use by a plurality of healthcare provider, receive electronic medication orders, the medication management facility configured to perform at least one of:
A method and system for communication between patients and pharmacy staff for quick clear efficient and error-free real-time communication with feedback and follow up capabilities in the provision, assessment, tracking, and documenting the decisions.
A determination of drug interaction information associated with the electronic medication orders;
A system to facilitate the user through the database data for development of correct ingredients;
A system to facilitate the user through the database data for development of correct strength of required ingredients;
A system to facilitate the user through the database data for development of correct combination of required ingredients;
A system to facilitate the user through the database data for development of correct base;
A system to facilitate the user through the database data for development of correct quantity of ingredients;
A system to facilitate the user through the database data for development of correct dosage of ingredients;

2: A medication validation, price estimation and and billing facility in electronic communication with the medication management facility, the medication validation and billing facility configured to receive the electronic medication orders, provide information generating an order, seeking approval from the customer, transmitting order, determining the right ingredients and quantity regarding formulary compliance, the drug allergy information, and the drug interaction information, and further comprising an interface for providing output to the a pharmacist.
